(19) United States
(12) Patent Application Publication
(10) Pub. No.: US 2017/0176445 A1
(43) Pub. Date: Jun. 22, 2017

(54) METHOD FOR DETERMINING THE LEVEL OF HYPOXIA IN A TUMOR

(71) Applicants: Universiteit Maastricht, Maastricht (NL); Academisch Ziekenhuis Maastricht, Maastricht (NL)

(72) Inventors: Kasper Mathias Antoon ROUSCHOP, Maastricht (NL); Tom Gertrudis Hubertus KEULERS, Maastricht (NL)

(73) Assignees: Universiteit Maastricht, Maastricht (NL); Academisch Ziekenhuis Maastricht, Maastricht (NL)

(21) Appl. No.: 15/118,397

(22) PCT Filed: Feb. 11, 2015
(86) PCT No.: PCT/EP2015/052854

§ 371 Date: Aug. 11, 2016

(30) Foreign Application Priority Data
Feb. 11, 2014 (EP) 14154698.6

(51) Int. Cl. G01N 33/574; C07K 16/18

(52) U.S. Cl. CPC G01N 33/57488; C07K 16/18; G01N 2800/7038

(57) ABSTRACT

The invention is in the field of medicine and molecular therapeutics. The invention provides means and measures for diagnosis and treatment, in particular for diagnosis and treatment of tumors, more in particular for determining the level of hypoxia in a tumor and for reducing the risk of metastasis of a tumor in a subject. The invention comprises a method wherein a level of exosome-associated GABARAPL1 is determined in a bodily fluid of the subject and wherein an elevated level of exosome-associated GABARAPL1 is indicative of an increased level of hypoxia of the tumor. The invention also comprises an antibody directed against GABARAPL1 for use in the treatment or prevention of cancer.

### FIELD OF THE INVENTION

The invention is in the field of medicine and molecular therapeutics. The invention provides means and measures for diagnosis and treatment, in particular for diagnosis and treatment of tumors, more in particular for determining the level of hypoxia in a tumor and for reducing the risk of metastasis of a tumor in a subject.

### BACKGROUND OF THE INVENTION

Local recurrence and distant metastasis frequently occur after radiation therapy for cancer and can be fatal. Evidence obtained from radiochemical and radiobiological studies has revealed these problems to be caused, at least in part, by a tumor-specific microenvironment, hypoxia.

It has extensively been described that the majority of solid human tumors contain regions that are poorly oxygenated, a very heterogeneous and dynamic feature [1]. In addition to the classical perception of tumor hypoxia that is caused by limitation in oxygen diffusion (chronic hypoxia), tumors are characterized by the presence of regions displaying periodic cycling in oxygenation (acute hypoxia) [2], which can account for a large proportion of the hypoxic cells [3].

From a clinical point of view, means of reducing the hypoxic fraction of tumors is highly desired since low oxygenation of tumors is associated with poor outcome in multiple cancer types [4], independent of treatment modality [5]. The observed effect on local control is most likely caused by the resistance of hypoxic cells to both chemo- and radiotherapy. Additionally there appears to be an association between hypoxia and the occurrence of metastasis [7]. It has been proposed that tumor hypoxia contributes to malignancy e.g. through activation of epithelial to mesenchymal transition [8-11] and formation of the pre-metastatic niche through secretion of specific microvesicles, extracellular vesicles; hereafter called exosomes [12-20]. Exosomes are known to mediate the transfer of mRNAs and microRNAs from one cell to the other [88].

Importantly, in head and neck squamous cell carcinoma (HNSCC) evidence of the therapeutic benefit of hypoxia modification is strongest [21], indicating that hypoxia modification does not only influence local control, but also overall outcome.

Tumor hypoxia is known to activate autophagy [24]. Autophagy is a pro-survival mechanism that allows cells to recycle its constituents and address alternative sources for energy production. Previously, we have shown that autophagy is primarily localized in the hypoxic fraction of the tumor and that hypoxia itself is a very powerful trigger for induction of autophagy [24].

It was observed that autophagy is activated within 30 min after hypoxia exposure [24]. Inhibiting autophagy (genetically or pharmacologically using chloroquine) sensitized cells to hypoxia, reduced the viable hypoxic fraction in tumors and subsequently sensitized these tumors to irradiation [24].

Hypoxia is thus a hallmark of solid cancers, which increases resistance to chemo- and radiotherapy and is associated with increased malignancy. The golden standard for assessment of autophagy and crucial in the autophagic process is microtubule-associated protein light chain 3b (LC3b). This protein is now widely used to monitor autophagy [84] and antibodies are commercially available (Santa Cruz Biotechnology).

Present methods for the detection of hypoxia, however, suffer from the disadvantage that they can only be performed ex vivo, requiring a biopsy or cell sample to be taken from the diseased tissue. Apart from being painful and costly, such a procedure also has inherent risks.

Non-invasive methods for determining tumor hypoxia are rare, US2003/0044862 describes such a method based on determining osteopontin levels in bodily fluids of patients. However, the level of this marker in hypoxic tumors was relatively close to the normal level in healthy volunteers (p=0.002, t-test) and therefore of limited use when applied to a single patient.

### SUMMARY OF THE INVENTION

We previously observed that GABARAPL1, an LC3b homologue, is upregulated during hypoxic exposure [85].

We have now surprisingly found that GABARAPL1 is not involved in the general execution of autophagy. This is based on the observation that knockdown of GABARAPL1 did not affect the autophagic flux during normal or stress conditions. Additionally, we found that GABARAPL1, unlike LC3b, is not essential for cell survival during hypoxia. Interestingly, knockdown of GABARAPL1 resulted in accumulation of mitochondria, suggesting a more specific role in mitochondrial autophagy, mitophagy. Moreover, mitochondrial uncoupling induced by CCCP (a potent mitophagy activator), induced GABARAPL1 expression.

GABARAPL1 mRNA is known to be present in exosomes obtainable from mast cells [88] among more than a thousand other RNA molecules. It was now surprisingly found that GABARAPL1 protein was expressed on exosomes and that these exosomes could be detected extracellular and in circulation of cancer patients. This finding allows the development of a method for determining the level of hypoxia in a tumor of a subject, wherein a level of exosome-associated GABARAPL1 is determined in a bodily fluid of the subject and wherein an elevated level of exosome-associated GABARAPL1 is indicative of an high level of hypoxia of the tumor, also commonly referred to as presence of hypoxia.

The finding also has therapeutic applications. We found that the risk on metastasis and therewith the number of metastases are reduced when GABARAPL1-containing exosomes are contacted with specific binding agents, such as antibodies against GABARAPL1. Our data as presented herein show that the mere binding of antibodies to their target GABARAPL1 protein is sufficient to provide the effects as described herein. In an alternative embodiment, the immune complexes comprising the antibody and the GABARAPL1-containing exosomes were removed from circulation. This may be performed in a number of ways, for instance by targeting GABARAPL1-containing exosomes with specific binding molecules such as antibodies. Complexes between antibodies and GABARAPL1-containing exosomes may then be cleared from circulation by conventional mechanisms, such as the natural clearance of immune complexes from circulation. In another embodiment, GABARAPL1-containing exosomes may be cleared from circulation by plasmapheresis.

The invention thus relates to a binding agent specific for GABARAPL1 for use in the treatment of cancer, more in particular by reducing the risk of metastasis of a tumor in a subject. This may be accomplished by targeting the GABARAPL1-containing exosomes in circulation or in a preferred embodiment by decreasing the level of circulating GABARAPL1-containing exosomes in the subject.

Without wanting to be bound by theory, we hypothesize that by reducing the number of GABARAPL1-containing exosomes or blocking GABARAPL1 function on the exosomes, the number of pre-metastatic niches is reduced.

It is rapidly becoming evident that the formation of tumor-promoting pre-metastatic niches in secondary organs adds a previously unrecognized degree of complexity to the challenge of curing metastatic disease. Primary tumor cells orchestrate pre-metastatic niche formation through secretion of a variety of cytokines and growth factors that promote mobilization and recruitment of bone marrow-derived cells to future metastatic sites.

Hypoxia within the primary tumor, and secretion of specific microvesicles such as exosomes, are emerging as important processes and vehicles for tumor-derived factors to modulate pre-metastatic sites. It has also come to light that reduced immune surveillance is a novel mechanism through which primary tumors create favorable niches in secondary organs. Reference 86 provides an overview of the current understanding of underlying mechanisms of pre-metastatic niche formation and highlights the common links as well as discrepancies between independent studies. [86].

In one specific embodiment, the invention provides a method for reducing the risk of metastasis of a tumor, by reducing the level or amount of circulating GABARAPL1-containing exosomes.

We have found that GABARAPL1-containing exosomes contain a number of pro-angiogenic compounds, such as VEGF. In this sense, the effect of treatment as described in this application may be similar to that of avastine/bevacizumab (Roche/Genentech).

Therapeutic methods as described herein may lead to a decreased risk of metastasis of the tumor and an increased susceptibility to therapeutic interventions.

### DETAILED DESCRIPTION OF THE INVENTION

To determine GABARAPL1 function in vivo, we created doxycycline inducible HT29 GABARAPL1 knockdown cells. Implantation of GABARAPL1 knockdown cells resulted in a delay in tumor formation and progression. GABARAPL1 knockdown in established tumors did not affect tumor progression. Additionally, GABARAPL1 knockdown directly induced after a single dose of irradiation (10 Gy), delayed tumor regrowth as compared to the control tumors.

GABARAPL1 is an LC3b-homologue. Several LC3b homologues have been identified, including gamma-aminobutyric-acid-type-A (GABAA)-receptor associated protein (GABARAP), GEC1/GABARAP-like1 (GABARAPL1) and GATE16/GABARAPL2. Similar to LC3b, these proteins are highly conserved among species ranging from mammals to yeast [29]. The function of all of these homologues remains unclear, but several studies suggest a common role in intracellular targeting of receptors or other proteins. All homologues require ubiquitin-like processes that catalyzes covalent conjugation of phosphatidylethanolamine (PE) to the protein [30]. Most homologues have been implicated in autophagy, although some more convincingly than others. However, functional studies towards the role of GABARAPL proteins in autophagy have not yet been performed.

We report herein our observation that GABARAPL1 is not involved in autophagy. We found that mRNA and protein production of GABARAPL1 was induced during hypoxia. We also detected GABARAPL1 upregulation in hypoxic tumor regions as assessed by pimonidazole co-localization. Interestingly, GABARAPL1 displays a punctuate pattern indicating that GABARAPL1 associates with vesicles during hypoxia.

Surprisingly, GABARAPL1 knockdown had no effect on autophagic flux and, unlike LC3b, did not accumulate upon blocking autophagy, whereas LC3b is partially degraded during autophagy. This suggests that GABARAPL1 is not crucially involved in formation and/or processing of autophagosomes. This was supported by confocal microscopy and immunofluorescent staining of LC3b and GABARAPL1 in cells. Although there seems to be a co-expression of LC3b and GABARAPL1 on several vesicles, the majority of vesicles display a mismatch. During hypoxia, GABARAPL1 displays a remarkable pattern with foci-formation and congregation at the cells' perimeter, a pattern that is not observed for LC3b. These data together suggest that the homologues GABARAPL1 and LC3b are required for distinct processes.

Furthermore, fluorescent LC3b or GABARAPL1 expression in cells revealed distinct trafficking. LC3b vesicles appeared relatively immobile whereas GABARAPL1 vesicles were transported from the perinuclear region (site of production) to the perimeter. This strongly suggests that GABARAPL1 vesicles mediate exocytosis during hypoxia exposure.

To confirm this, we used click-chemistry detection of de novo synthesized and secreted proteins and immunoblot analysis. We observed an overall reduction in protein secretion in hypoxic GABARAPL1-deficient cells. Remarkably, this difference was not observed under normal oxygen conditions and shows that GABARAPL1 plays a role in protein secretion specifically under hypoxic conditions. In parallel, we have upscaled this procedure and have isolated de novo synthesized and secreted proteins for identification by mass spectrometry. SILAC-analysis revealed a shift in protein secretion. Analysis of the top-20 changed (lower in GABARAPL1-deficient MCF7 and HT29 cells) secreted proteins, revealed that 12 of the 20 proteins that were changed most, were proteins previously detected in- or associated with exosomes, including CD81, a general exosome marker (table 1). These findings are in line with the vesicular localization of GABARAPL1 and trafficking to the membrane.

As used herein, the term “exosome” refers to cell-derived vesicles that are present in many and perhaps all biological fluids, including blood, plasma, serum, urine, and cultured medium of cell cultures. The reported diameter of exosomes is between 30 and 100 nm, which is larger than LDL, but much smaller than for example red blood cells. Exosomes are either released from the cell when multivesicular bodies fuse with the plasma membrane or they are released directly from the plasma membrane. It is becoming increasingly clear that exosomes have specialized functions and play a key role in, for example, coagulation, intercellular signaling, and waste management. Exosomes can potentially be used for prognosis, therapy, and biomarkers for health and disease.

To further test if GABARAPL1 is involved in exosome secretion, mCherry-labeled GABARAPL1 was co-transfected with eGFP-labeled CD63 or CD81, both well recognized exosomal marker proteins. We found that GABARAPL1 partially co-localized with both markers. This co-localization was even more pronounced for CD81 (>90% vesicle colocalization, than CD63 (5-10% vesicle colocalization), suggesting the assembly of GABARAPL1 to subsets of exosomes. Furthermore, isolation of secreted exosomes revealed co-expression of GABARAPL1 and the exosomal marker CD81.

Importantly, a subset of exosomes secreted by hypoxia-exposed cells and exosomes derived from serum of cancer patients express endogenous GABARAPL1 at the outside of the exosome as assessed by immunofluorescent staining of intact exosomes. This is important as it allows antibody mediated targeting of GABARAPL1 exosomes.

The invention therefore relates to a method for determining the level of hypoxia in a tumor of a subject, wherein a level or amount of exosome-associated GABARAPL1 is determined in a bodily fluid of the subject and wherein an elevated level of exosome-associated GABARAPL1 is indicative of an increased level of hypoxia of the tumor. Such a method may advantageously be performed in a bodily fluid such as blood, serum or plasma.

To determine the presence or absence of tumor hypoxia in a patient according to the methods of this invention, the level of GABARAPL1-associated exosomes is detected by methods such as those illustrated herein.

The term “elevated level” is to be interpreted as a level above a predetermined level. In a specific embodiment of a method according to the invention, a level of GABARAPL1-associated exosomes is typically compared to a predetermined value that is capable of distinguishing between hypoxic tumors and non-hypoxic, such as oxic tumors in a specified patient population. The predetermined value may be an empirically determined value or range of values determined from test measurements on groups of patients with a particular class of tumor, e.g., head and neck, breast, or colon cancer. Alternatively, the predetermined value may be based on values measured in a particular patient over a period of time. The skilled person is well aware of methods by which a predetermined value for GABARAPL1-associated exosomes levels may be empirically determined in patients or normal individuals.

In another preferred embodiment, the predetermined value is determined using a Receiver Operator Curve. This method may be used to arrive at the most accurate cut-off value, taking into account the false positive rate and the false negative rate of the diagnostic assay.

The invention may therefore also be described as providing a method for determining the level of hypoxia in a tumor of a subject, wherein a level or amount of exosome-associated GABARAPL1 is determined in a bodily fluid of the subject and wherein an level of exosome-associated GABARAPL1 above a predetermined reference value is indicative of an increased level of hypoxia of the tumor.

Kits and compounds for determining the level or amount of exosome-associated GABARAPL1 are also provided herein. Such kits may comprise a binding agent for exosome-associated GABARAPL1, such as an antibody specific for GABARAPL1 and a calibration means for comparing the level of exosome-associated GABARAPL1 with a predetermined value.

TABLE 1
Top 20 less secreted proteins during hypoxia
in GABARAPL1 knockdown cells and association
with exosomes and metastasis formation.

| | | | Detected in | |
|---|---|---|---|---|
| | | Exosome | exosomes derived/ | Metastasis |
| | Protein name | associated | isolated from | associated |
| 1 | Heat shock protein | Yes | Mast cells | [37, 38] |
| | HSP 90-beta | | | |
| 2 | CD81 antigen | Yes | a general | [39] |
| | | | exosome marker, | |
| | | | bladder-, | |
| | | | colorectal-, | |
| | | | melanoma-, | |
| | | | prostate-cancer | |
| | | | cells, B-cells, | |
| | | | mesenchymal | |
| | | | stem cells, | |
| | | | T-cells, | |
| | | | throphoblasts | |
| | | | detected in | |
| | | | ascites, breast | |
| | | | milk, saliva, | |
| | | | urine. | |
| 3 | Receptor-type | | | [40] |
| | tyrosine-protein | | | |
| | phosphatase F | | | |
| 4 | Alpha-actinin-4 | | | |
| 5 | Stanniocalcin-2 | Yes | Mesenchymal stem | [41-43] |
| | | | cell | |
| 6 | Heterogeneous | Yes | B-cell, breast | |
| | nuclear ribonucleo- | | cancer, | |
| | proteins | | colorectal | |
| | A2/B1 | | cancers | |
| 7 | Sulfhydryl | Yes | Colorectal | [44] |
| | oxidase 1 | | cancer, | |
| | | | detected in | |
| | | | urine | |
| 8 | Protein S100-A11 | Yes | Colorectal | [45, 46] |
| | | | cancer | |
| 9 | Keratin, type I | | Bladder-, | |
| | cytoskeletal 18 | | colorectal | |
| | | | cancer | |
| 10 | Transferrin | | | |
| | receptor protein 1 | | | |
| 11 | Laminin subunit | Yes | Colorectal | [47] |
| | alpha-5 | | cancer | |
| 12 | Heat shock protein | Yes | Bladder- breast | [48-58] |
| | 27 beta-1 | | cancer, saliva, | |
| | | | Urine | |
| 13 | Nucleophosmin | Yes | Detected in | [59, 60] |
| | | | urine | |
| 14 | Acidic leucine- | | | |
| | rich nuclear | | | |
| | phosphoprotein | | | |
| | 32 member A | | | |
| 15 | Mucin-5B | | | |
| 16 | UPF0364 protein | | | |
| | C6orf211 | | | |
| 17 | Alanine-tRNA | Yes | B-cells | |
| | ligase, cytoplasmic | | | |
| 18 | Stress-induced- | Yes | Bladder cancer | [61, 62] |
| | phosphoprotein 1 | | | |
| 19 | Fructose-1,6- | Yes | Detected in | |
| | bisphosphatase 1 | | urine | |
| 20 | Endostatin | | | |

Also preferred is a method as described above, additionally comprising the steps of isolating an exosome and detecting the amount, number or level of exosome-associated GABARAPL1.

The method is preferably performed on subjects with a solid tumor. The level of exosome-associated GABARAPL1 may advantageously be determined using a method selected from the group consisting of immunoblotting, ELISA or flow cytometry.

The functional role of GABARAPL1 in mediating exosome secretion is unknown. GABARAPL1 may play a role in vesicle formation, trafficking or cargo selection. Based on the observation that GABARAPL1 is expressed only on a subset of exosomes and thus not for general exosome biogenesis or transport; It is most likely that GABARAPL1 is required for cargo selection of exosomes, specifically in hypoxic cells.

Exosomes, like autophagosomes, consist of a lipid bilayer membrane surrounding a small cytosol but are devoid of cellular organelles. They can contain various molecular constituents of their cell of origin, including proteins and nucleic acid material (eg mRNA or miRNA) [63]. Over the past period exosomes have been identified as a method of mediating cell-cell communication that influences major tumor associated mechanisms, such as epithelial to mesenchymal transition [64], cancer stemness, angiogenesis [13] and metastasis [65]. Interestingly, given the association between tumor hypoxia, exosomes and the occurrence of metastasis [18, 66], we found that a number of proteins, that have been associated with exosomes, have been directly implicated in the occurrence of metastasis (table 1). These data show that inhibition of GABARAPL1-associated exosome secretion leads to a reduction in metastasis.

To determine if the decrease in exosomes, observed in the GABARAPL1 deficient cells also decreased secretion of pro-angiogenic factors, we used antibody arrays that allowed us to assess multiple of these factors. Indeed, several factors (VEGF, CXCL16, Angiogenin and PDGF) were found to be secreted less in the GABARAPL1 deficient cells after hypoxia. In line, xenograft implantation of GABARAPL1 deficient cells in the flanks of nude mice, displayed decreased tumor growth (FIG. 1A). Indeed, reduced growth of the GABARAPL1 deficient tumors was associated with less vascularization (FIG. 1B) and increased tumor hypoxia (FIG. 1C).

Hence, the invention also relates to a method of reducing the risk of metastasis of a tumor in a subject, wherein the level of circulating GABARAPL1-containing exosomes is decreased in the subject. The method may also be equally suited for reducing angiogenesis or the vascularisation of the tumor or for reducing the volume of the tumor. Hence, the invention also relates to a method for inhibiting, decreasing or preventing angiogenesis or neovascularisation of a tumor. The invention also relates to a method for sensitizing tumors to therapy, preferably radiotherapy. The invention also relates to a method for reducing the metastatic potential through inhibition of exosome secretion or otherwise decreasing the level or amount of circulating GABARAPL1-associated exosomes.

There are many suitable ways known to a skilled person for reducing the level of exosome-associated GABARAPL1. For example, this may be accomplished by contacting the GABARAPL1-containing exosomes with a binding agent, such as an antibody. In a preferred embodiment, such a binding agent may be administered intravascular. In another preferred embodiment, the tumor is a solid tumor.

As used herein, the term ‘elevated level” or increased level” or “increased amount” or equivalent refers to a level or amount that is higher than in a normal subject. Vise versa, the term ‘lowered level” or decreased level” or “decreased amount” or equivalent refers to a level or amount that is lower or less than in a normal subject wherein a normal subject is a subject without a tumor. The term “less” or “lower” in this respect is to be interpreted preferably as substantially less, significantly less or more than 10% less than the level or amount in a normal person. More than 10% in this respect may be more than 20%, 30%, 40% or even more than 50% less. In a preferred embodiment, the level of GABARAPL1-associated exosomes is reduced by more than 50%, such as 60, 70, 80, 90 or even 95% or more, such as 96, 97, 98, 99 or even 100%.

### LEGEND TO THE FIGURES

FIG. 1: (A) Tumor growth, (B) vessel density and (C) hypoxic fraction of control (shSCR) and GABARAPL1 knockdown (shGABARAPL1) tumors.

FIG. 2: (A) tube formation by HUVEC after exposure to exosomes derived from control (SCR) or GABARAPL1 deficient tumour cells exposed to hypoxia. (B) inhibition of hypoxia-associated-exosome tube formation by anti-GABARAPL1 antibody. (C) anti-GABARAPL1 is only capable of reducing tube formation when initiated by exposure to exosomes.

FIG. 3: MDA-MB-231 adherence to HUVEC monolayers. HUVEC pre-exposure to exosomes facilitates adherence of tumor cells. This effect can be abrogated by GABARAPL1 blocking antibodies.

FIG. 4: MDA-MB-231 lung metastases. Control and GABARAPL1 knockdown cells were implanted orthotopically in nude mice. After reaching 1500 mm3, the lungs were excised and the number of metastasis were assessed.

FIG. 5: Correlation of plasma GABARAPL1 exosomes with HX4 high volume in NSCLC patients.

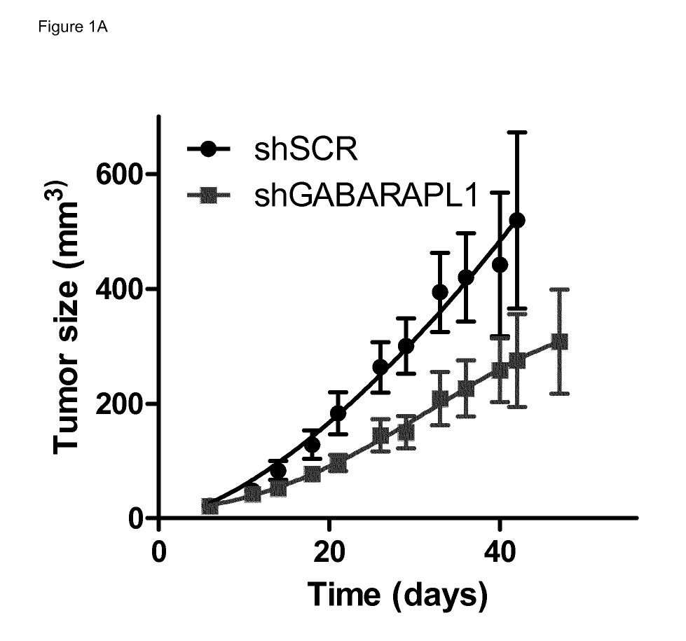

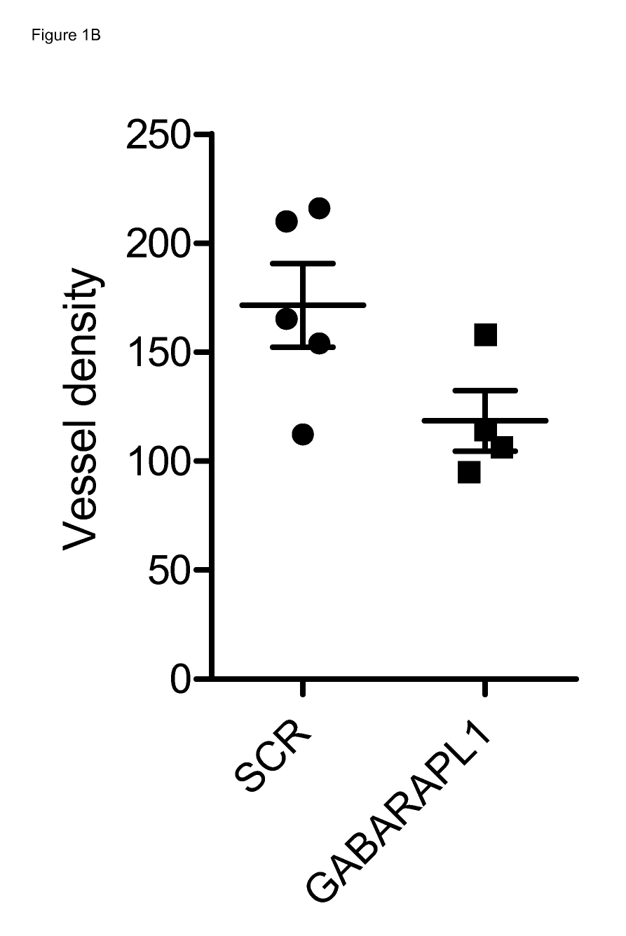

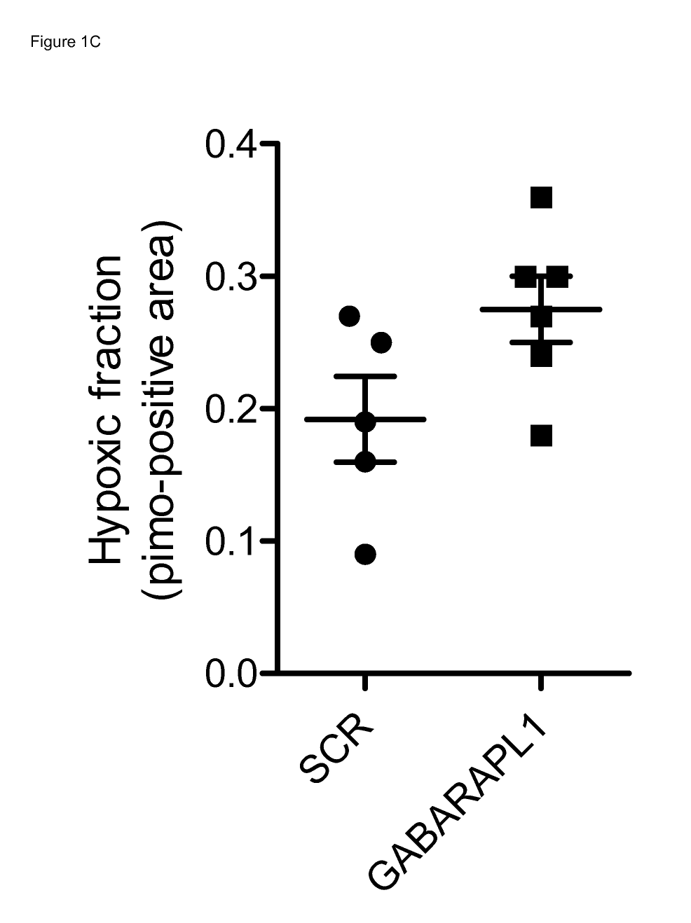

### EXAMPLES

### Example 1: Cell Culturing

MCF7 (mammary adenocarcinoma), HT29 (colorectal adenocarcinoma), and u87 (glioblastoma) cell lines were cultured in RPMI and DMEM (GE healthcare) growth media respectively with 10% FCS in a 5% CO2 incubator at 37° C. For hypoxia exposure, cells were transferred to ananoxic (0.0% O2) culture chamber (MACS VA500 microaerophilic workstation; Don Whitley Scientific). Lipofectamine 2000 (Invitrogen) was used for plasmid transfections.

### Example 2: Cloning/Virus Production

Lentiviral pTRIPz vectors encoding Tet-inducible shRNA-GABARAPL1 and shRNA-Scrambled were purchased from Open Biosystems. For the production of viral vectors, HEK293T cells were transfected with Bug of VSV-G/envelope (addgene 8454) and pCMV(delta) R8.74/packaging (addgene 22036) and pTRIPz-GABARAPL1 or pTRIPz-SCR plasmids. Subsequently, virus containing media were collected and 2, 3 and 4 days after transfection and aliquoted. MCF7 and HT29 cells were transduced, and after selection with puromycin (4 μg/mL) for 10 days, cells were pooled and analyzed. To induce knockdown, Doxycycline (1 μg/mL, Sigma) was added 72 hours prior to experiments.

### Example 3: Exosome Production Medium

To avoid contamination of bovine exosomes, fetal calf serum was depleted of exosomes by ultracentrifugation at 100.000×g over night (16 h). Before addition to the medium, the exosome-depleted serum was filter-sterilized by a 0.22 um filter (Millipore).

### Example 4: Exosome Isolation

Cells were grown until they reached 80% of confluency. To induce exosome secretion, cells were exposed to anoxia for 24 h. Exosomes were isolated from the media by differential ultracentrifugation (Beckman Coulter, sw41Ti rotor) with increasing centrifugation speeds. To remove large dead cells en cellular debris, samples were centrifuged at 300×g and 16.000×g for 5 and 30 minutes respectively. The pellet was thrown away. The final supernatant was centrifuged at 100.000×g for 90 minutes. The exosome-containing pellet was washed with a large amount of PBS and centrifuged again at 100.000×g for 90 minutes. All centrifugation steps were done at 4° C.

### Example 5: Staining Endogenous GABARAPL1

Endogenous GABARAPL1 was stained by applying GABARAPL1 antibody (protein tech group, 1:50) and secondary antibody to the resuspended isolated exosomes. Exosomes were pelleted and washed with PBS. Mounted in mounting medium and visualized by fluorescence microscopy.

### Example 6: Fusion Proteins/Colocalization

GABARAPL1 and LC3B PCR-fragments were subcloned (EcoRI/XhoI, New England Biolabs) into eGFP-C1 and mCherry-C1 backbone vectors (Clontech). eGFP and mCherry were fused at the N-terminal site.

### Example 7: Exosome Pulldown

Isolated exosomes were incubated with GABARAPL1 antibody (1:50) for 6 hours at 4° C. Subsequently, blocked magnetic dynabeads (2 hours 1% BSN PBS-Tween, RT) were added to the exosome containing sample and incubated overnight at 4° C. at a head over head shaker. After incubation, magnetic pulldown was performed and beads were washed 3 times with PBS. Protein content was visualized by westernblot.

### Example 8: Angiogenesis Array

HT29 and MCF7 GABARAPL1 knockdown and control cells were exposed to anoxia (0% 02) for 24 hours and medium was collected. Before applying conditioned medium to the angiogenesis array (Human Angiogenesis Antibody Array, Catalog #ARY007), medium was centrifuged for 10 minutes 300×g. Angiogenesis array was performed according to the manufacturers manual.

### Example 9: Mass Spec Sample Preparation

HT29 and MCF7 GABARAPL1 knockdown and control cells were exposed to anoxia (0.0% 02) for 24 hours. Conditioned medium (DMEM and RPMI respectively, 0% FCS) was collected and concentrated with Amicon® Ultra 3K filters. Samples were visualized by Page gel electrophoresis or mass spec analysis.

### Example 10: Endothelial Tube Formation Assay

Human umbilical vessel cells (HUVEC) (20.000 cells) were seeded in a matrigel coated (BD-biosciences, 50 μL/well) 96-well. If applicable, cells were exposed to isolated exosomes of hypoxia (O2<0.02%) exposed tumor cells with/without anti-GABARAPL1 antibody (proteintechgroup, #10010-AP-1). Tube development was assessed after 16 hours exposure at 37 C.

### Example 11: Tumor Cell Adherence

HUVEC cells were grown as monolayer in 24-well format. When indicated, HUVEC monolayers were exposed for 16 hours to isolated exosomes of hypoxia (O2<0.02%) exposed tumor cells with/without anti-GABARAPL1 antibody (proteintechgroup, #10010-AP-1). 20.000 GFP-expressing (eGFP-C1, clontech) MDA-MB-231 tumor cells were added to the monolayers. After exposure for the indicated timepoints, the monolayers were extensively washed with PBS. The number of adhering cells was quantified after trysinization by flow cytometry.

### Example 12: Metastasis Development

Control cells and GABARAPL1 knockdown cells were generated using pTRIPZ vectors. One million MDA-MB-231 cells were implanted in the fat-pad of female nude mice (nu/nu NMRI, Charles River). Tumor growth was assessed using caliper measurements. After reaching a primary tumor volume of 15003, the animals were killed and the lungs examined for metastasis. The lung nodules were counted manually.

### Example 13: The Role of GABARAPL1 in Exosome Function

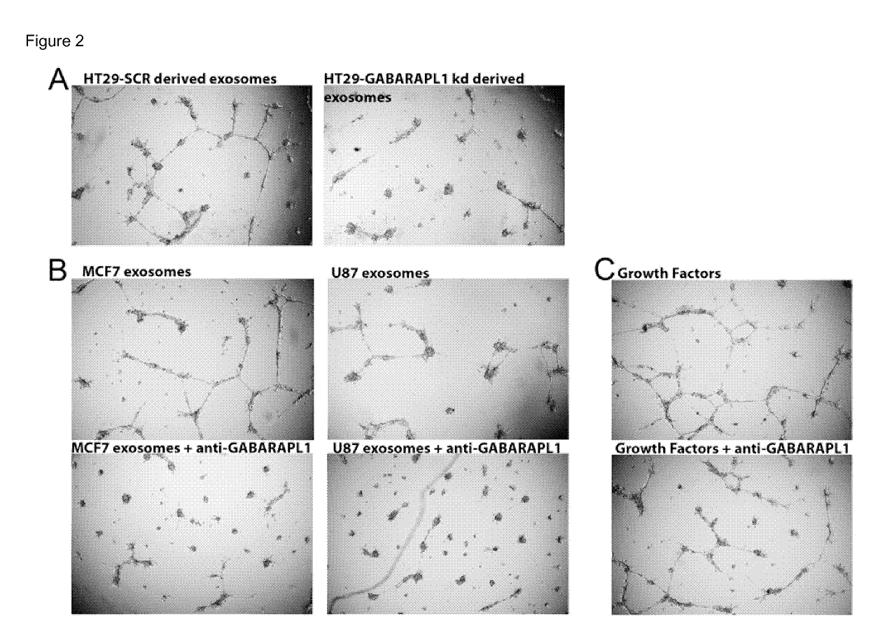

To assess if GABARAPL1 has a direct role in hypoxia-associated exosome function, we used a capillary tube formation assay in vitro. Exposure of human umbilical cord vessel endothelial cells (HUVEC) to exosome derived from hypoxia-exposed HT29, MCF7 and U87 cells (FIG. 2A) were capable of inducing tube formation in vitro. Interestingly, compared to their respective controls, exosome derived from GABARAPL1 knockdown proved less capable of inducing tube formation (FIG. 2A). Blocking GABARAPL1 using specific antibodies efficiently blocked exosome-induced tube formation (FIG. 2B). The GABARAPL1 antibody effective against exosome-induced tube formation, was unable to prevent tube formation when growth factors (VEGF, FGF-2, IGF, EGF) were added to the culture medium directly (FIG. 2C), indicating the specificity of anti-GABARAPL1 action through inhibition of exosome function and that GABARAPL1-exosome function can be inhibited by antibody targeting.

### Example 14: GABARAPL1 Exosomes Facilitate Adherence of Tumor Cells to Endothelium

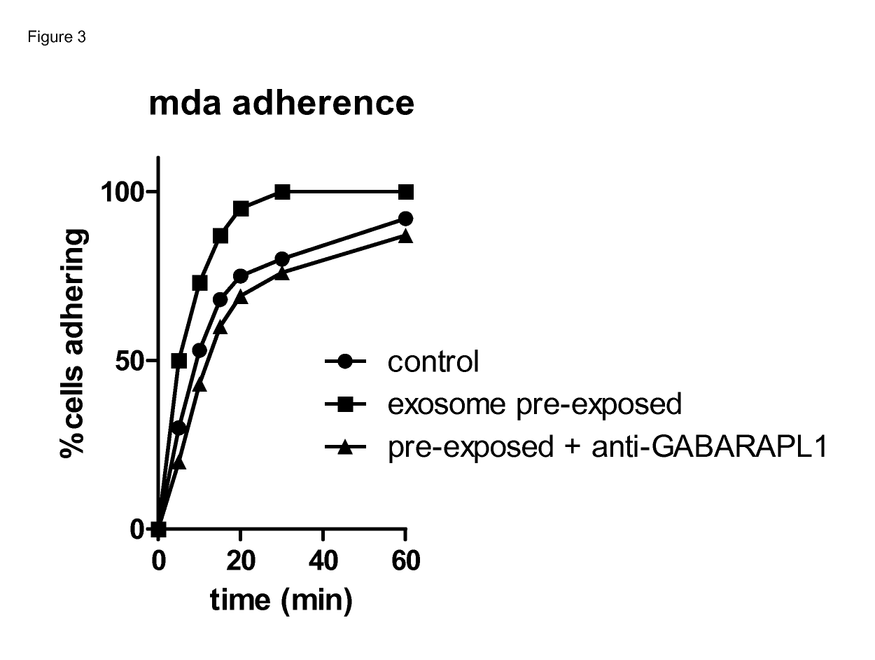

As shown herein, GABARAPL1 exosomes have a profound effect on enodothelial cells. This effect can be inhibited through the use of GABARAPL1 blocking antibodies. In the progress of metastasis, adherence of metastasizing tumor cells to the vessel endothelial cells is essential. To determine whether GABARAPL1 exosomes facilitate adherence of tumor cells to endothelium, HUVEC monolayers were grown and adherence of tumor cells (fluorescently labeled MDA-MB-231) was assessed by flow cytometry. Under normal conditions, 30% of the added tumor cells adhered within 5 minutes to the endothelial cells. After 1 hour, 92% of the seeded tumor cells adhered. Surprisingly, pre-exposure of endothelial cells to isolated exosomes facilitated adherence of tumor cells (50% adhered within 5 minutes and 100% after 30 minutes), which could be inhibited by pre-exposure to exosomes in combination with GABARAPL1 blocking antibodies (20% adherence after 5 minutes and 76% after 30 minutes) (FIG. 3).

### Example 15: GABARAPL1 Reduction Reduces Metastasis Development

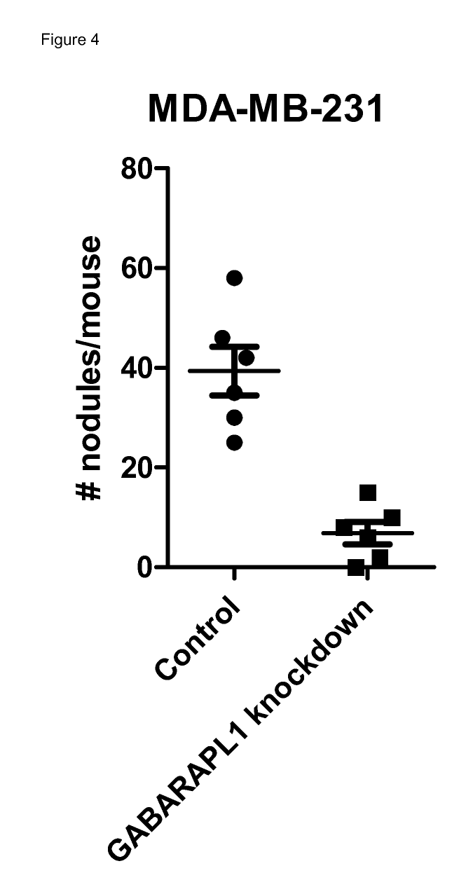

GABARAPL1 exosomes facilitate tumor cell adhesion to endothelial cells, and important step in tumor cell extravasation and development of metastasis. To determine if tumors in the absence of GABARAPL1 show reduced capacity of metastasis development, MDA-MB-231 cells were generated with GABARAPL1 knockdown. Both control and GABARAPL1 knockdown cells were implanted orthotopically in nude mice. When the primary tumors reached 1500 mm3 in size, the animals were killed and the lungs of the animals were examined for metastasis development. On average, control tumors lead to the development of 39.3 metastasis, whereas GABARAPL1 knockdown tumors lead to development of 6.8 metastasis per animal (FIG. 4).

### Example 16: The Number of GABARAPL1 Exosomes in Blood Correlate with Tumor Hypoxia

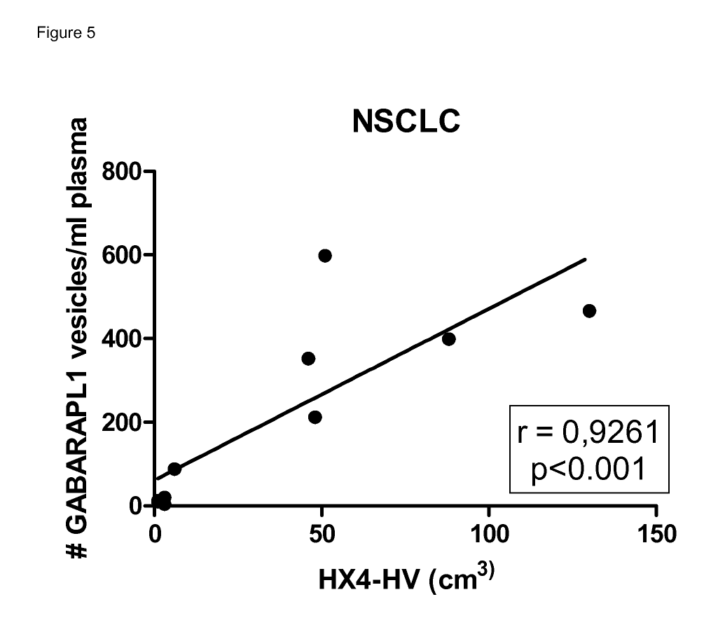

In blood of healthy volunteers, no GABARAPL1 exosomes could be detected, whereas in blood of cancer patients GABARAPL1 exosomes are frequently observed. To determine if GABARAPL1 exosomes in blood can be used as biomarker for tumor hypoxia, we analyzed plasma of non-small cell lung cancer (NSCLC) patients that were assessed for the degree of tumor hypoxia by HX4 PET-scanning (Zegers et al.). Exosomes were isolated from 1 ml of plasma. GABARAPL1 exosomes were visualized by immunochemical staining using anti-GABARAPL1 antibodies. Interestingly the number of GABARAPL1-exosomes correlated with HX4 high volume (r=0.9261, P<0.001, FIG. 5).

### REFERENCES

1. Brown, J. M. and W. R. Wilson, Exploiting tumour hypoxia in cancer treatment. Nat Rev Cancer, 2004. 4(6): p. 437-47.
2. Magagnin, M. G., M. Koritzinsky, and B. G. Wouters, Patterns of tumor oxygenation and their influence on the cellular hypoxic response and hypoxia-directed therapies. Drug Resist Updat, 2006.
3. Durand, R. E. and C. Aquino-Parsons, Non-constant tumour blood flow—implications for therapy. Acta Oncol, 2001. 40(7): p. 862-9.
4. Wouters, B. G., et al., Targeting hypoxia tolerance in cancer. Drug Resist Updat, 2004. 7(1): p. 25-40.
5. Hockel, M., et al., Association between tumor hypoxia and malignant progression in advanced cancer of the uterine cervix. Cancer Res, 1996. 56(19): p. 4509-15.
6. Nordsmark, M., et al., Prognostic value of tumor oxygenation in 397 head and neck tumors after primary radiation therapy. An international multi-center study. Radiother Oncol, 2005. 77(1): p. 18-24.
7. Brizel, D. M., et al., Tumor oxygenation predicts for the likelihood of distant metastases in human soft tissue sarcoma. Cancer Res, 1996. 56(5): p. 941-3.
8. Gort, E. H., et al., Hypoxic regulation of metastasis via hypoxia-inducible factors. Curr Mol Med, 2008. 8(1): p. 60-7.
9. Gort, E. H., et al., The TWIST1 oncogene is a direct target of hypoxia-inducible factor-2alpha. Oncogene, 2008. 27(11): p. 1501-10.
10. Theys, J., et al., E-Cadherin loss associated with EMT promotes radioresistance in human tumor cells. Radiother Oncol, 2011. 99(3): p. 392-7.
11. Yang, M. H. and K. J. Wu, TWIST activation by hypoxia inducible factor-1 (HIF-1): implications in metastasis and development. Cell Cycle, 2008. 7(14): p. 2090-6.
12. Filipazzi, P., et al., Recent advances on the role of tumor exosomes in immunosuppression and disease progression. Semin Cancer Biol, 2012. 22(4): p. 342-9.
13. Grange, C., et al., Microvesicles released from human renal cancer stem cells stimulate angiogenesis and formation of lung premetastatic niche. Cancer research, 2011. 71(15): p. 5346-56.
14. Jung, T., et al., CD44v6 dependence of premetastatic niche preparation by exosomes. Neoplasia, 2009. 11(10): p. 1093-105.
15. Peinado, H., S. Lavotshkin, and D. Lyden, The secreted factors responsible for pre-metastatic niche formation: old sayings and new thoughts. Semin Cancer Biol, 2011. 21(2): p. 139-46.
16. Salomon, C., et al., Exosomal signaling during hypoxia mediates microvascular endothelial cell migration and vasculogenesis. PloS one, 2013. 8(7): p. e68451.
17. Sceneay, J., et al., Hypoxia-driven immunosuppression contributes to the pre-metastatic niche. Oncoimmunology, 2013. 2(1): p. e22355.
18. Sceneay, J., M. J. Smyth, and A. Moller, The pre-metastatic niche: finding common ground. Cancer metastasis reviews, 2013.
19. Svensson, K. J., et al., Hypoxia triggers a proangiogenic pathway involving cancer cell microvesicles and PAR-2-mediated heparin-binding EGF signaling in endothelial cells. Proc Natl Acad Sci USA, 2011. 108(32): p. 13147-52.
20. Wong, C. C., et al., Hypoxia-inducible factor 1 is a master regulator of breast cancer metastatic niche formation. Proc Natl Acad Sci USA, 2011. 108(39): p. 16369-74.
21. Overgaard, J., Hypoxic modification of radiotherapy in squamous cell carcinoma of the head and neck—A systematic review and meta-analysis. Radiother Oncol, 2011.
22. Rouschop, K. M., et al., Deregulation of cap-dependent mRNA translation increases tumour radiosensitivity through reduction of the hypoxic fraction. Radiother Oncol, 2011. 99(3): p. 385-91.
23. Rouschop, K. M., et al., PERK/eIF2alpha signaling protects therapy resistant hypoxic cells through induction of glutathione synthesis and protection against ROS. Proceedings of the National Academy of Sciences of the United States of America, 2013. 110(12): p. 4622-7.
24. Rouschop, K. M., et al., The unfolded protein response protects human tumor cells during hypoxia through regulation of the autophagy genes MAP1 LC3B and ATGS. J Clin Invest, 2010. 120(1): p. 127-41.
25. Sarkar, S., et al., Hypoxia induced impairment of NK cell cytotoxicity against multiple myeloma can be overcome by IL-2 activation of the NK cells. PloS one, 2013. 8(5): p. e64835.
26. Klionsky, D. J., et al., Guidelines for the use and interpretation of assays for monitoring autophagy in higher eukaryotes. Autophagy, 2008. 4(2): p. 151-75.
27. Dubois, L. J., et al., Preclinical evaluation and validation of [18F]HX4, a promising hypoxia marker for PET imaging. Proc Natl Acad Sci USA, 2011. 108(35): p. 14620-5.
28. van Loon, J., et al., PET imaging of hypoxia using [18F]HX4: a phase I trial. Eur J Nucl Med Mol Imaging, 2010. 37(9): p. 1663-8.
29. Coyle, J. E. and D. B. Nikolov, GABARAP: lessons for synaptogenesis. Neuroscientist, 2003. 9(3): p. 205-16.
30. Chakrama, F. Z., et al., GABARAPL1 (GEC1) associates with autophagic vesicles.

Autophagy, 2010. 6(4).
31. Kabeya, Y., et al., LC3, GABARAP and GATE16 localize to autophagosomal membrane depending on form-II formation. J Cell Sci, 2004. 117(Pt 13): p. 2805-12.
32. Schwarten, M., et al., Nix directly binds to GABARAP: a possible crosstalk between apoptosis and autophagy. Autophagy, 2009. 5(5): p. 690-8.
33. Novak, I., et al., Nix is a selective autophagy receptor for mitochondrial clearance. EMBO Rep. 11(1): p. 45-51.
34. Betin, V. M. and J. D. Lane, Caspase cleavage of Atg4D stimulates GABARAP-L1 processing and triggers mitochondrial targeting and apoptosis. J Cell Sci, 2009. 122(Pt 14): p. 2554-66.
35. Ballot, G., et al., Hypoxia-induced autophagy is mediated through hypoxia-inducible factor induction of BNIP3 and BNIP3L via their BH3 domains. Mol Cell Biol, 2009. 29(10): p. 2570-81.
36. Zhang, J. and P. A. Ney, Role of BNIP3 and NIX in cell death, autophagy, and mitophagy. Cell Death Differ, 2009. 16(7): p. 939-46.
37. Biaoxue, R., et al., Upregulation of Hsp90-beta and annexin A1 correlates with poor survival and lymphatic metastasis in lung cancer patients. Journal of experimental & clinical cancer research: CR, 2012. 31: p. 70.
38. Wang, J., et al., High expression of heat shock protein 90 is associated with tumor aggressiveness and poor prognosis in patients with advanced gastric cancer. PloS one, 2013. 8(4): p. e62876.
39. Mazzocca, A., F. Liotta, and V. Carloni, Tetraspanin CD81-regulated cell motility plays a critical role in intrahepatic metastasis of hepatocellular carcinoma. Gastroenterology, 2008. 135(1): p. 244-256 el.
40. Trojan, L., et al., Identification of metastasis-associated genes in prostate cancer by genetic profiling of human prostate cancer cell lines. Anticancer Res, 2005. 25(1A): p. 183-91.
41. Kita, Y., et al., STC2: a predictive marker for lymph node metastasis in esophageal squamous-cell carcinoma. Annals of surgical oncology, 2011. 18(1): p. 261-72.
42. Pena, C., et al., STC1 expression by cancer-associated fibroblasts drives metastasis of colorectal cancer. Cancer research, 2013. 73(4): p. 1287-97.
43. Volland, S., et al., Stanniocalcin 2 promotes invasion and is associated with metastatic stages in neuroblastoma. International journal of cancer. Journal international du cancer, 2009. 125(9): p. 2049-57.
44. Katchman, B. A., et al., Expression of quiescin sulfhydryl oxidase 1 is associated with a highly invasive phenotype and correlates with a poor prognosis in Luminal B breast cancer. Breast cancer research: BCR, 2013. 15(2): p. R28.
45. Meding, S., et al., Tissue-based proteomics reveals FXYD3, S100A11 and GSTM3 as novel markers for regional lymph node metastasis in colon cancer. The Journal of pathology, 2012.
46. Nipp, M., et al., S100-A10, thioredoxin, and S100-A6 as biomarkers of papillary thyroid carcinoma with lymph node metastasis identified by MALDI imaging. Journal of molecular medicine, 2012. 90(2): p. 163-74.
47. Hibino, S., et al., Identification of an active site on the laminin alpha5 chain globular domain that binds to CD44 and inhibits malignancy. Cancer research, 2004. 64(14): p. 4810-6.
48. Chen, J., et al., Proteome analysis of gastric cancer metastasis by two-dimensional gel electrophoresis and matrix assisted laser desorption/ionization-mass spectrometry for identification of metastasis-related proteins. Journal of proteome research, 2004. 3(5): p. 1009-16.
49. Gibert, B., et al., Targeting heat shock protein 27 (HspB1) interferes with bone metastasis and tumour formation in vivo. Br J Cancer, 2012. 107(1): p. 63-70.
50. Guo, K., et al., Regulation of HSP27 on NF-kappaB pathway activation may be involved in metastatic hepatocellular carcinoma cells apoptosis. BMC Cancer, 2009. 9: p. 100.
51. Nagaraja, G. M., P. Kaur, and A. Asea, Role of human and mouse HspB1 in metastasis. Curr Mol Med, 2012. 12(9): p. 1142-50.
52. Shiota, M., et al., Hsp27 regulates epithelial mesenchymal transition, metastasis, and circulating tumor cells in prostate cancer. Cancer Res, 2013. 73(10): p. 3109-19.
53. Song, H. Y., et al., [Proteomic analysis on metastasis-associated proteins of hepatocellular carcinoma tissues]. Zhonghua Gan Zang Bing Za Zhi, 2005. 13(5): p. 331-4.
54. Song, H. Y., et al., Proteomic analysis on metastasis-associated proteins of human hepatocellular carcinoma tissues. J Cancer Res Clin Oncol, 2006. 132(2): p. 92-8.
55. Wei, L., et al., Hsp27 participates in the maintenance of breast cancer stem cells through regulation of epithelial-mesenchymal transition and nuclear factor-kappaB. Breast Cancer Res, 2011. 13(5): p. R101.
56. Zhang, D., L. L. Wong, and E. S. Koay, Phosphorylation of Ser78 of Hsp27 correlated with HER-2/neu status and lymph node positivity in breast cancer. Molecular cancer, 2007. 6: p. 52.
57. Zhao, L., et al., Differential proteomic analysis of human colorectal carcinoma cell lines metastasis-associated proteins. J Cancer Res Clin Oncol, 2007. 133(10): p. 771-82.
58. Zhao, M., et al., Increased expression of heat shock protein 27 correlates with peritoneal metastasis in epithelial ovarian cancer. Reprod Sci, 2012. 19(7): p. 748-53.
59. Coutinho-Camillo, C. M., et al., Nucleophosmin, p53, and Ki-67 expression patterns on an oral squamous cell carcinoma tissue microarray. Hum Pathol, 2010. 41(8): p. 1079-86.
60. Liu, Y., et al., Expression of nucleophosmin/NPM1 correlates with migration and invasiveness of colon cancer cells. J Biomed Sci, 2012. 19: p. 53.
61. Walsh, N., et al., RNAi knockdown of Hop (Hsp70/Hsp90 organising protein) decreases invasion via MMP-2 down regulation. Cancer Lett, 2011. 306(2): p. 180-9.
62. Walsh, N., et al., Identification of pancreatic cancer invasion-related proteins by proteomic analysis. Proteome Sci, 2009. 7: p. 3.
63. van den Boom, J. G., et al., Exosomes as nucleic acid nanocarriers. Advanced drug delivery reviews, 2013. 65(3): p. 331-5.
64. Holzel, M., A. Bovier, and T. Tuting, Plasticity of tumour and immune cells: a source of heterogeneity and a cause for therapy resistance? Nature reviews. Cancer, 2013. 13(5): p. 365-76.
65. Park, J. E., et al., Hypoxic tumor cell modulates its microenvironment to enhance angiogenic and metastatic potential by secretion of proteins and exosomes. Molecular & cellular proteomics: MCP, 2010. 9(6): p. 1085-99.
66. Azmi, A. S., B. Bao, and F. H. Sarkar, Exosomes in cancer development, metastasis, and drug resistance: a comprehensive review. Cancer metastasis reviews, 2013.
67. King, H. W., M. Z. Michael, and J. M. Gleadle, Hypoxic enhancement of exosome release by breast cancer cells. BMC Cancer, 2012. 12: p. 421.
68. Kucharzewska, P., et al., Exosomes reflect the hypoxic status of glioma cells and mediate hypoxia-dependent activation of vascular cells during tumor development. Proceedings of the National Academy of Sciences of the United States of America, 2013. 110(18): p. 7312-7.
69. Karreman, M. A., et al., Optimizing immuno-labeling for correlative fluorescence and electron microscopy on a single specimen. Journal of structural biology, 2012. 180(2): p. 382-6.
70. Schaaf, M. B., et al., The autophagy associated gene, ULK1, promotes tolerance to chronic and acute hypoxia. Radiotherapy and oncology: journal of the European Society for Therapeutic Radiology and Oncology, 2013.
71. Jutten, B., et al., EGFR overexpressing cells and tumors are dependent on autophagy for growth and survival. Radiotherapy and oncology: journal of the European Society for Therapeutic Radiology and Oncology, 2013.
72. Liu, Q., et al., TH-302, a hypoxia-activated prodrug with broad in vivo preclinical combination therapy efficacy: optimization of dosing regimens and schedules. Cancer Chemother Pharmacol, 2012. 69(6): p. 1487-98.
73. Meng, F., et al., Molecular and cellular pharmacology of the hypoxia-activated prodrug TH-302. Mol Cancer Ther, 2012. 11(3): p. 740-51.
74. Sun, J. D., et al., Selective tumor hypoxia targeting by hypoxia-activated prodrug TH-302 inhibits tumor growth in preclinical models of cancer. Clin Cancer Res, 2012. 18(3): p. 758-70.
75. Jemal, A., et al., Global cancer statistics. CA: a cancer journal for clinicians, 2011. 61(2): p. 69-90.
76. Bacia, K., S. A. Kim, and P. Schwille, Fluorescence cross-correlation spectroscopy in living cells. Nat Methods, 2006. 3(2): p. 83-9.
77. Vaupel, P., et al., Oxygenation of human tumors: evaluation of tissue oxygen distribution in breast cancers by computerized 02 tension measurements. Cancer Res, 1991. 51(12): p. 3316-22.
78. Fyles, A., et al., Tumor hypoxia has independent predictor impact only in patients with node-negative cervix cancer. Journal of clinical oncology: official journal of the American Society of Clinical Oncology, 2002. 20(3): p. 680-7.
79. Pitson, G., et al., Tumor size and oxygenation are independent predictors of nodal diseases in patients with cervix cancer. International journal of radiation oncology, biology, physics, 2001. 51(3): p. 699-703.
80. Sundfor, K., H. Lyng, and E. K. Rofstad, Tumour hypoxia and vascular density as predictors of metastasis in squamous cell carcinoma of the uterine cervix. British journal of cancer, 1998. 78(6): p. 822-7.
81. Milosevic, M., et al., Tumor hypoxia predicts biochemical failure following radiotherapy for clinically localized prostate cancer. Clinical cancer research: an official journal of the American Association for Cancer Research, 2012. 18(7): p. 2108-14.
82. Rouschop, K. M., et al., Autophagy is required during cycling hypoxia to lower production of reactive oxygen species. Radiother Oncol, 2009. 92(3): p. 411-6.
83. Klionsky, D. J., et al., Guidelines for the use and interpretation of assays for monitoring autophagy. Autophagy, 2012. 8(4): p. 445-544.
84. Klionsky, D. J. et al., Guidelines for the use and interpretation of assays for monitoring autophagy in higher eukaryotes. Autophagy. 2008 February; 4(2):151-75.
85. Keulers et al., Abstractbook 13th international Wolfsberg meeting on molecular Radiation Biology/oncology, Ermatingen Switzerland, Jun. 22-24, 2013, p 88.
86. Sceneay, J. et al., The pre-metastatic niche: finding common ground Cancer Metastasis Rev. 2013 December; 32(3-4):449-64.
87. Zegers C M, van Elmpt W, Reymen B, Even A J, Troost E G, Oilers M C, Hoebers F J, Houben R M, Eriksson J, Windhorst A D, Mottaghy F M, De Ruysscher D, Lambin P. In Vivo Quantification of Hypoxic and Metabolic Status of NSCLC Tumors Using [18F]HX4 and [18F]FDG-PET/CT Imaging. Clin Cancer Res. 2014 Dec. 15; 20(24):6389-97.
88. Valadi, H. et al., Nature Cell Biol. (2007) 9: 654-659 and supplementary information, table S2.

1. A method for determining a level of hypoxia in a tumor of exosome-associated GABARAPL1 in a subject having a tumor, the method comprising:
a determining the level of exosome-associated GABARAPL1 in a bodily fluid of the subject.

2. The method according to claim 1, wherein the bodily fluid is blood, serum or plasma.

3. The method according to claim 1 further comprising isolating an exosome and determining the level of exosome-associated GABARAPL1.

4. The method according to claim 1, wherein the tumor is a solid tumor.

5. The method according to claim 1, wherein the level of exosome-associated GABARAPL1 is determined using a method selected from the group consisting of immunoblotting, ELISA, and flow cytometry.

6. A method of treating a subject having a tumor, the method comprising:
administering to the subject an antibody directed against GABARAPL1.

7. The method according to claim 6, wherein the risk of metastasis of the tumor in the subject is reduced.

8. The method according to claim 6, wherein GABARAPL1-containing exosomes are removed from circulation.

9. The method according to claim 6, wherein the antibody is administered intravascularly.

10. The method according to claim 6, wherein angiogenesis or neovascularization of the tumor is inhibited, decreased, or prevented.

11. The method according to claim 6, wherein the tumor is a solid tumor.

12. The method according to claim 1, further comprising comparing the determined level of exosome-associated GABARAPL1 to a control level.
